(19) United States
(12) Patent Application Publication
(10) Pub. No.: US 2013/0072918 A1
(43) Pub. Date: Mar. 21, 2013

(54) HANDPIECE INTENDED PARTICULARLY FOR MEDICAL LASER APPLICATIONS

(71) Applicant: elexxion AG, Radolfzell (DE)

(72) Inventor: Olaf Schafer, Singen (DE)

(73) Assignee: ELEXXION AG, Radolfzell (DE)

(21) Appl. No.: 13/667,684

(22) Filed: Nov. 2, 2012

Related U.S. Application Data
(62) Division of application No. 12/304,290, filed on Dec. 11, 2008, now abandoned.

(30) Foreign Application Priority Data
Jun. 13, 2006 (DE) 102006027624.8

(51) Int. Cl. A61B 18/22

(52) U.S. Cl. 606/16

(57) ABSTRACT

A handpiece for a medical laser comprising an inner channel (6, 8, 8.1) into which a laser fiber (7) is fitted and protrudes with a free end portion of length (1) from a channel mouth (10), the length (1) of the laser fiber (7) protruding from the channel mouth (10) is adjustable.

### BACKGROUND OF THE INVENTION

The invention relates to a handpiece intended particularly for medical laser applications, comprising an inner channel into which a laser fiber is fitted, with a length of a free end section of the laser fiber protruding out of a channel opening.

These days, lasers are used for operations or treatments in a number of medical fields, with reference being made only in an exemplary manner to eye operations, prostate treatments and the use in dentistry. The laser is mainly used for cutting and shaving purposes, but, in particular, also for coagulating tissue. In the first case, a laser beam is generally emitted over a certain distance so that the actual laser or the corresponding handpiece does not come into direct contact with the area around the operating site. By way of example, such a handpiece is shown in DE 699 20 236 T2. However, the penetration depth and scattering in biological tissue are critical obstacles for this laser radiation.

However, a laser can be used very expediently, in particular for treating biological tissue, if the laser fiber is brought into contact with the tissue. The cutting action is effected by the distal end of the laser fiber which, heated by the laser radiation, vaporizes the touched tissue area. The bleeding is also stemmed by the corresponding coagulation. It was found to be disadvantageous in this case that the tip of the laser fiber becomes contaminated and also worn down when in direct contact with the tissue. The contamination by charred blood particles and tissue particles reduces or prevents emission of the radiation at the tip of the fiber. It is for this reason that the laser fiber has to be replaced very frequently, especially since it is exposed to mechanical damage by its long protruding length. This is expensive and requires a lot of time.

To counteract this disadvantage, DE 42 09 926 A1 proposes that an alternating or simultaneous application of laser radiation for cutting and coagulating is attained by means of two concentrically arranged optical waveguides into which laser radiation with a variable intensity distribution profile is coupled so that the greater part of the beam power is incident to the respectively desired extent on either the inner or outer fiber, or incident on both. That is to say the laser fiber is divided into an inner fiber and an outer fiber, with a laser fiber having lens optics present in the handpiece being assigned to these two fibers. This laser fiber arranged in the interior is at such a distance from the combination of inner fiber and outer fiber that either the inner fiber or the outer fiber is supplied with the laser radiation.

Apart from the fact that this device is difficult to operate, the region of the laser fiber protruding from the channel opening remains subject to mechanical wear and tear, and the abovementioned contamination.

It is the object of the present invention to reduce wear and tear of the laser fiber tip protruding from the channel opening in a simple manner.

### SUMMARY OF THE INVENTION

The object is achieved by the length of the laser fiber protruding from the channel opening being changeable.

This means that during a medical application, the laser fiber only protrudes from the channel opening by the smallest possible amount. Hence, it is subject to less mechanical wear and tear since it is more difficult to break a short fiber section than a long fiber section. Furthermore, only this short fiber section is contaminated. If this fiber section is so contaminated that no beam can pass through it, it can, for example, be mechanically broken off when it is outside the operating site. By means of these two advantages, the service life of currently used laser fibers can be substantially increased, and hence the costs can also be reduced.

In one exemplary embodiment, known single-piece handpieces could continue to be used and inserting a laser fiber into this handpiece could be restricted. In this case, the laser fiber would have to be designed such that it can be pushed through the handpiece bit by bit. This appears to be complicated, but is feasible.

On the other hand, in a preferred exemplary embodiment, the handpiece is intended to be designed such that its dimensions are changeable. This implies that the laser fiber and its lens optics remain in one place in the handpiece and hence the optics are not disturbed in any case. This ensures the functional reliability of the laser. The connection between the laser fiber and the optics is maintained without change.

The change of the dimensions of the laser can firstly be effected by the handpiece being produced from at least two parts, with the position of these parts with respect to one another being changeable. Two possibilities are preferred for this positional change: a first possibility is that two parts are axially displaced with respect to one another. That is to say the handpiece is virtually stretched, as a result of which part of the laser fiber protruding from the channel opening is pulled into the inner channel.

For this purpose, the two parts are preferably connected by a latching connection, with it being possible for this latching connection to be designed in an arbitrary manner. In a preferred exemplary embodiment, the handpiece is divided into a connection piece and a front piece. These are connected by a plug connection, that is to say the connection piece uses a plug-in sleeve to engage in a push-on sleeve of the front piece. Of course, this arrangement can also be reversed. In order to now simplify the latching connection, a plurality of crenellated latching rings are shaped on the periphery of the plug-in sleeve and interact with elastic elements, in particular rubber rings, which are held in the push-on sleeve by corresponding annular grooves. If the connection piece and front piece are now pulled apart, the rubber rings squeeze past the crenellated latching rings and then expand into the intermediate spaces between the latching rings such that the front piece is fixed there with respect to the connection piece.

A further possible latching tooth system could be implemented by assigning a latching tooth system, which interacts with a notch lever to be operated from the outside, to, for example, the front piece or else the connection piece. Said notch lever is preferably designed as a tilt lever, with the tilt lever being held in the latching position by means of a corresponding pressure spring.

It would also be feasible that provision is made for a thread toothing in this area so that, for example, the connection piece could be screwed out of the front piece. However, this results in the risk of twisting the laser fiber and hence this is undesirable. In this case, it would be better to pull the laser fiber out of the inner channel, rotate the thread and thereafter reinsert the laser fiber. However, this is cumbersome and requires time.

However, such twisting of two parts of the handpiece with respect to one another is possible in the vicinity of the opening of the laser fiber because twisting a screw insert there does not influence the laser fiber much. Hence, the arrangement of a second exemplary embodiment would be possible there, in which the screw insert with a male thread meshes with a female thread in the front piece. This exemplary embodiment should be selected in particular if the change of length of the fiber piece protruding from the channel opening has to be determined very precisely, since this can be achieved particularly well by the screw insert.

In order to fix the screw insert in its desired position, provision is made for a clamping element such as a set screw or the like.

### BRIEF DESCRIPTION OF THE DRAWINGS

Further advantages, features and details of the invention emerge from the following description of preferred exemplary embodiments and from the drawing, in which:

FIG. 1 shows a longitudinal section through a handpiece for medical laser applications according to the invention;

FIG. 2 shows a longitudinal section through the handpiece according to FIG. 1 in another position used;

FIG. 3 shows a partly illustrated longitudinal section through a region of a front piece of a handpiece for medical laser applications in a further embodiment;

FIG. 4 shows a section of a longitudinal section according to FIG. 3 in another position used; and

FIG. 5 shows a section of a longitudinal section of a further exemplary embodiment of a handpiece for medical laser applications.

### DETAILED DESCRIPTION

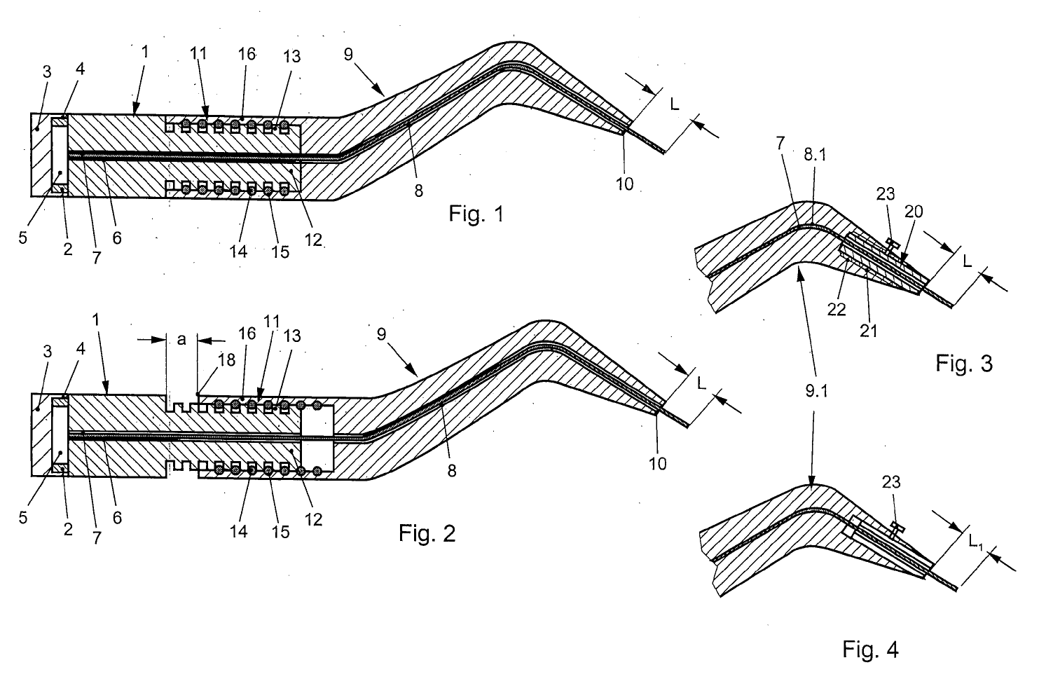

According to FIG. 1, a handpiece for medical laser applications according to the invention comprises a cylindrical connection piece 1, which on the left-hand side forms an annular flange 2 provided with a male thread not identified in any more detail. This male thread interacts with a female thread (likewise not identified in any more detail) of a cap 3 which is screwed onto the annular flange 2. Together with the connection piece 1, the cap 3 forms an annular space 4 for holding a fiber connection 5, or lens optics, which is adjoined by a laser fiber 7 guided in an axial channel 6.

The axial channel 6 merges into a channel 8 which extends through a front piece 9 of the handpiece. The channel 8 is guided through the front piece 9 in arcs, depending on the curvature of the front piece 9. Together with the axial channel 6, the channel 8 forms an inner channel for guiding the laser fiber 7 which emerges from a channel opening 10 at the tip of the front piece 9 and which protrudes by a length l out of the channel opening 10.

The front piece 9 is connected to the connection piece 1 by a latching connection 11. For this purpose, a plurality of crenellated latching rings 13 are formed on the periphery of a plug-in sleeve 12 of the connection piece 1, said latching rings 13 interacting with rubber rings 14 which are in corresponding annular grooves 15 in the inner surface of a push-on sleeve 16 formed by the front piece 9.

The present invention operates as follows, and is explained in connection with FIG. 2:

In FIG. 1, the push-on sleeve 16 is completely pushed over the plug-in sleeve 2 such that the laser fiber 7 protrudes from the channel opening 10 with its greatest length l. If, for example at the beginning of a laser treatment, this length l is now intended to be reduced such that only a small area of the tip of the laser fiber 7 is contaminated or worn down, then the connection piece 1 and front piece 9 are pulled apart by pulling, with the rubber rings 14 being squeezed by the latching rings 13 and then being able to expand again into the intermediate spaces between the latching rings 13. If, for example, two crenellated latching rings 13 are skipped by the rubber rings 14, as is illustrated in FIG. 2, then this results in a distance a between an inner edge 17 of the connection piece 1 and an end edge 18 of the push-on sleeve 16. At the same time, the length of the end of the laser fiber 7 protruding from the channel opening 10 reduces to a length l.

If, over time, a greater length of laser fiber is required, the front piece 9 is pushed further over the plug-in sleeve 12, latching ring by latching ring, such that the desired length is always protruding from the channel opening.

In the case of a further exemplary embodiment of the invention according to FIGS. 3 and 4, a screw insert 20 is intended to be inserted into the tip of the front piece 9. This screw insert 20 has a male thread 21 which interacts with a female thread 22 in the front piece 9.1. The screw insert 20 also forms a channel section 8.1 for guiding the laser fiber 7.

If the length l is intended to be reduced to the length l₁, as shown in FIG. 4, a clamping screw 23 is loosened and the screw insert 20 can be unscrewed a little from the front piece 9.1.

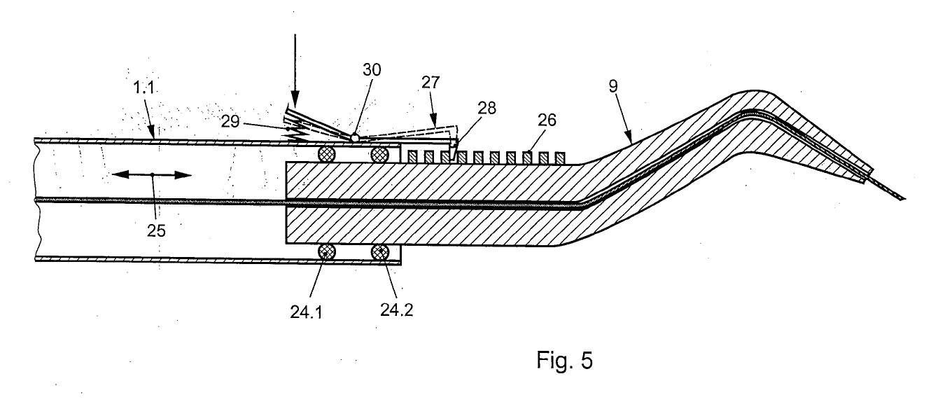

In a further exemplary embodiment of the invention according to FIG. 5, the front piece 9 is pushed into a tube-shaped connection piece 1.1. Two sliding rings 24.1 and 24.2 are provided between the front piece 9 and the connection piece 1.1 for the purposes of sealing and mounting. The front piece 9 can be displaced in the tube-shaped connection piece 1.1 according to the double-headed arrow 25.

According to the invention, a latching tooth system 26 is placed on the front piece 1 and interacts with a notch lever 27. This notch lever 27 is designed as a tilt lever and has a latching tooth 28 on its one free end facing the latching tooth system 26, while on its other free end a pressure spring 29 braces itself against said free end and the connection piece 1.1. Between the two free ends, the notch lever 27 has an articulated connection 30 with the connection piece 1.1.

If a finger pushes against the end above the pressure spring 29, then the notch lever 27 tilts about the articulated connection 30 and the latching tooth 28 disengages from the latching tooth system 26. Hence the front piece 9 can be displaced within the tube-shaped connection piece 1.1 in the direction of the double-headed arrow 25.

The possibility of the illustrated devices for changing the length l being provided together on a handpiece is of course also within the scope of the invention.

1-12. (canceled)

13. A handpiece for a medical laser comprising:
a connection piece and a front piece;
latching means between said connection piece and said front piece for moving the connection piece and front piece relative to each other;
a first channel in said connection piece and a second channel in said front piece;
a laser fiber fixed relative to the first channel and passing through the second channel and protruding therefrom a length (l), wherein said latching means allows the front piece to move axially relative to the connection piece and laser fiber for varying the length (l) of the laser fiber protruding from the second channel, the latching means comprises a latching tooth system provided on the front piece and an actuable tilt lever attached to the connection piece which engages in the latching tooth system.

14. The handpiece as claimed in claim 13, wherein the tilt lever is actuated by a spring.
